(19) United States
(12) Patent Application Publication
(10) Pub. No.: US 2024/0416103 A1
(43) Pub. Date: Dec. 19, 2024

(54) A CARDIAC PUMP

(71) Applicant: CALON CARDIO-TECHNOLOGY LTD., Swansea (GB)

(72) Inventors: Matthew Paul BIGINTON, Swansea (GB); Robyn WILLIAMS, Swansea (GB)

(21) Appl. No.: 18/703,015

(22) PCT Filed: Sep. 29, 2022
(86) PCT No.: PCT/EP2022/077151

§ 371 Date: Apr. 19, 2024

(30) Foreign Application Priority Data
Oct. 20, 2021 (GB) 2114999.2

(51) Int. Cl. A61M 60/148; A61M 60/178; A61M 60/422; A61M 60/82

(52) U.S. Cl. CPC A61M 60/148; A61M 60/178; A61M 60/422; A61M 60/82

(57) ABSTRACT

A cardiac pump includes a housing, a rotor extending along a rotational axis of the cardiac pump between first and second axial ends of the rotor, and a bearing assembly which supports the rotor within the cardiac pump housing for rotation about the rotational axis. The rotor includes a recess formed in an axial end face of the rotor. The housing includes a protruding portion extending axially into the recess. The bearing assembly includes: a first magnet assembly provided on a portion of the housing radially outward of the recess formed in the rotor, a second magnet assembly provided on a portion of the rotor radially outward of the protruding portion of the housing, and a third magnet assembly provided on the protruding portion of the housing. The first, second and third magnet assemblies at least partially overlap one another in a direction parallel with the rotational axis.

### TECHNICAL FIELD

The present disclosure relates to a blood pump, such as a cardiac pump and is particularly, although not exclusively, concerned with a cardiac pump configured to operate at subcritical rotor speeds.

### BACKGROUND

Advanced heart failure is a major global health problem resulting in many thousands of deaths each year and those with the disease endure a very poor quality of life. The treatment options for advanced heart failure, for example, drug therapy and cardiac resynchronization (pacemakers), have generally proved unsuccessful and the only option remaining for the patients is heart transplantation. Unfortunately, the number of donor hearts available only meets a fraction of the demand, leaving many people untreated.

Ventricular Assist Devices (VAD) have been gaining increased acceptance over the last decade as an alternative therapy to heart transplantation. The use of VADs has shown that, in most cases, once the device has been implanted, the disease progression is halted, the symptoms of heart failure are relieved, and the patient regains a good quality of life.

VADs can be considered as a viable alternative to treat heart failure and offer hope to the many thousands of heart failure patients for whom a donor heart will not be available.

In general terms, it is known to provide a cardiac pump, such as a VAD, that is suitable for implantation into a ventricle of a human heart. The most common type of these implantable pumps is a miniaturised rotary pump, owing to their small size and mechanical simplicity/reliability. Such known devices have two primary components: a cardiac pump housing, which defines a cardiac pump inlet and a cardiac pump outlet; and a cardiac pump rotor, which is housed within the cardiac pump housing, and which is configured to impart energy to the fluid.

A requirement for the cardiac pump, therefore, is a bearing system that rotatably supports the cardiac pump rotor within the cardiac pump housing. Bearings systems for cardiac pumps, and generally all rotating machines such as pumps and motors, ideally achieve the fundamental function of permitting rotation of the rotor, whilst providing sufficient constraint to the rotor in all other degrees of freedom, e.g. the bearing system must support the rotor axially, radially and in pitch/yaw.

Desirable functions of bearing systems generally may include low rates of wear and low noise and vibration, and in the case of blood pumps, elimination of features that trap blood, or introduce shear stress or heat in the blood.

In known devices, the cardiac pump rotor may be rotatably supported within the housing using one of a number of different types of bearing systems. In general, there are three types of bearing systems that are utilised in cardiac pumps.

Some cardiac pumps use blood-immersed contact bearings, for example a pair of plain bearings, to rigidly support the rotor within the housing. However, for such plain bearing systems it may be difficult to ensure that the rotor is perfectly entrapped within the contact bearings. Moreover, blood-immersed contact bearings of the prior art may be susceptible to proteinaceous and other biological deposition in the bearings, and also in the region proximate to the bearings and on supporting structures around the bearings.

Other cardiac pumps use non-contact hydrodynamic bearing systems, in which the rotor is supported on a thin film of blood. In order to produce the required levels of hydrodynamic lift, hydrodynamic bearing systems require small running clearances. As a consequence, blood that passes through those small running clearances may be subjected to high levels of shear stress, which may have a detrimental effect on the cellular components of the blood, for example, by causing haemolysis or platelet activation which may further lead to thrombosis.

Cardiac pumps may also employ non-contact magnetic bearing systems, in which the running clearances between the rotor and the housing may be designed such that large gaps can exist in the bearing and therefore shear-related blood damage in the bearing is reduced. However, it is common to use a passive magnetic bearing system in combination with another manner of support in at least one degree-of-freedom, for example active magnetic control, which may significantly increase the size and complexity of the design, and/or hydrodynamic suspension, which may increase the requirements with regard to manufacturing tolerances.

In existing cardiac pumps, the bearing system typically has a low stiffness, such that the rotor operates at a super-critical speed. The cardiac pumps must therefore be designed and constructed to tolerate the loads experiences when the rotor accelerates through its critical speed to reach the operating speed of the cardiac pump.

### STATEMENTS OF INVENTION

According to an aspect of the present disclosure, there is provided a blood pump, such as a cardiac pump comprising:
a housing;
a rotor extending along a rotational axis of the cardiac pump between first and second axial ends of the rotor; and
   a bearing assembly, wherein the bearing assembly is configured to support the rotor within the cardiac pump housing for rotation about the rotational axis, wherein the rotor comprises a recess formed in an axial end face of the rotor, wherein the housing comprises a protruding portion extending axially into the recess, and wherein the bearing assembly comprises:
a first magnet assembly provided on a portion of the housing radially outward of the recess formed in the rotor;
a second magnet assembly provided on a portion of the rotor radially outward of the protruding portion of the housing; and
a third magnet assembly provided on the protruding portion of the housing, wherein the first, second and third magnet assemblies at least partially overlap one another in a direction parallel with the rotational axis.

The first, second and third magnet assemblies may thereby be configured to support the rotor in a radial direction relative to the rotational axis.

The first axial end of the rotor may be an outlet end and the second axial end of the rotor may be an inlet end. The recess may be formed in the axial end face at the outlet end of the rotor. Alternatively, the recess may be formed in the axial end face at the inlet end of the rotor.

The first magnet assembly may comprise a plurality of magnets arranged to produce a stronger magnetic field adjacent the first magnet assembly in a radially inward direction and a weaker magnetic field adjacent to the first magnet assembly in a radially outward direction.

The first magnet assembly may comprise a half or full Halbach array of magnets, e.g. comprising 3 or more magnets, arranged to produce a stronger magnetic field adjacent the first magnet assembly in a radially inward direction and a weaker magnetic field adjacent to the first magnet assembly in a radially outward direction.

The first magnet assembly may comprise a first housing magnet, arranged such that a magnetic dipole moment of the first housing magnet is aligned with a first direction parallel with the rotational axis. The first magnet assembly may comprise a second housing magnet, arranged such that a magnetic dipole moment of the second magnet is aligned with a second direction opposite the first direction. The first magnet assembly may comprise a pole piece arranged between the first and second housing magnets. The first magnet assembly may comprise a pole piece between each magnet of the half or full halbach array. The first magnet assembly may comprise a third housing magnet.

The second magnet assembly may comprise a first rotor magnet, arranged such that a magnetic dipole moment of the first magnet is aligned with a first direction parallel with the rotational axis. The second magnet assembly may comprise a second rotor magnet adjacent the first rotor magnet, e.g. along the axis of rotation of the rotor, immediately adjacent the first rotor magnet, e.g. without a pole piece between the first and second magnets. The second rotor magnet may be arranged such that a magnetic dipole moment of the second magnet is aligned with a second direction opposite the first direction. The second magnet assembly may comprise a pole piece arranged between the first and second rotor magnets, e.g. along the rotational axis.

The third magnet assembly may comprise a plurality of magnets arranged to produce a stronger magnetic field adjacent the third magnet assembly in a radially outward direction and a weaker magnetic field adjacent to the third magnet assembly in a radially inward direction. For example, the third magnet assembly may comprise a half or full Halbach array of magnets arranged to produce a stronger magnetic field adjacent the third magnet assembly in a radially outward direction and a weaker magnetic field adjacent to the third magnet assembly in a radially inward direction.

The third magnet assembly may comprise a fourth housing magnet, arranged such that a magnetic dipole moment of the fourth housing magnet is aligned with a first direction parallel with the rotational axis. The third magnet assembly may comprise a fifth housing magnet, arranged such that a magnetic dipole moment of the fifth housing magnet is aligned with a second direction opposite the first direction. The third magnet assembly may comprise a pole piece arranged between the fourth and fifth housing magnets, e.g. along the rotational axis. The third magnet assembly may comprise a pole piece between each magnet of the half or full halbach array.

The bearing assembly may further comprise a fourth magnet assembly provided on the housing and a fifth magnet assembly provided on the rotor. The fourth and fifth magnet assemblies are spaced apart from the recess in the rotor in a direction parallel with the rotational axis. The fourth and fifth magnet assemblies may at least partially overlap one another in a direction parallel with the rotational axis.

The fourth and fifth magnet assemblies may be are arranged closer to the inlet end of the rotor than the first, second and third magnet assemblies. For example, the first, second and third magnet assemblies may be arranged to support, e.g. radially support, rotation of the rotor about the rotational axis at the outlet end and the fourth and fifth magnet assemblies may be arranged to support, e.g. radially support, rotation of the rotor about the rotational axis at the inlet end.

The cardiac pump may further comprise a magnetic drive assembly comprising one or more drive magnets for driving rotation of the rotor relative the housing. The one or more drive magnets may be arranged between the first, second and third magnet assemblies, and the fourth and fifth magnet assemblies.

The fourth magnet assembly may comprise a seventh housing magnet, arranged such that a magnetic dipole moment of the seventh housing magnet is aligned with a first direction parallel with the rotational axis. The fourth magnet assembly may comprise a eighth housing magnet. The eighth housing magnet may be adjacent the seventh housing magnet e.g. along the rotational axis. The eighth housing magnet may be immediately adjacent the seventh housing magnet, e.g. without a pole piece between the seventh and eighth housing magnets. The seventh housing magnet may be arranged such that a magnetic dipole moment of the seventh housing magnet is aligned with a second direction opposite the first direction.

The fifth magnet assembly may comprise a third rotor magnet, arranged such that a magnetic dipole moment of the third magnet is aligned with a first direction parallel with the rotational axis. The fifth magnet assembly may comprise a fourth rotor magnet adjacent the third rotor magnet e.g. along the rotational axis. The fourth rotor magnet may be immediately adjacent the third rotor magnet, e.g. without a pole piece between the third and fourth rotor magnets. The fourth rotor magnet may be arranged such that a magnetic dipole moment of the fourth rotor magnet is aligned with a second direction opposite the first direction.

The fifth magnet assembly may comprise a third rotor magnet, arranged such that a magnetic dipole moment of the third rotor magnet is aligned with a first direction parallel with the rotational axis. The fifth magnet assembly may comprise a fourth rotor magnet, arranged such that a magnetic dipole moment of the second magnet is aligned with a second direction opposite the first direction. The fifth magnet assembly may comprise a pole piece arranged between the third and fourth rotor magnets, e.g. along the rotational axis.

The fourth magnet assembly may comprise a plurality of magnets arranged to produce a stronger magnetic field adjacent the fourth magnet assembly in a radially inward direction and a weaker magnetic field adjacent to the fourth magnet assembly in a radially outward direction.

The fourth magnet assembly may comprise a half or full Halbach array of magnets arranged to produce a stronger magnetic field adjacent the fourth magnet assembly in a radially inward direction and a weaker magnetic field adjacent to the fourth magnet assembly in a radially outward direction. The fourth magnet assembly may comprise five or more magnets.

The fifth magnet assembly may comprise a plurality of magnets arranged to produce a stronger magnetic field adjacent the fifth magnet assembly in a radially outward direction and a weaker magnetic field adjacent to the fifth magnet assembly in a radially inward direction.

The fifth magnet assembly may comprise a half or full Halbach array of magnets arranged to produce a stronger magnetic field adjacent the fifth magnet assembly in a radially outward direction and a weaker magnetic field adjacent to the fifth magnet assembly in a radially inward direction.

The protruding portion of the housing may be configured to engage the rotor at the recess to form a plane bearing between the housing and the rotor at the first end of the rotor. The housing may be configured to engage the rotor at the second end of the rotor to form a further plane bearing between the housing and the rotor at the second end of the rotor.

To avoid unnecessary duplication of effort and repetition of text in the specification, certain features are described in relation to only one or several aspects or embodiments of the invention. However, it is to be understood that, where it is technically possible, features described in relation to any aspect or embodiment of the invention may also be used with any other aspect or embodiment of the invention.

### BRIEF DESCRIPTION OF THE DRAWINGS

For a better understanding of the present invention, and to show more clearly how it may be carried into effect, reference will now be made, by way of example, to the accompanying drawings, in which:

FIG. 1 is a perspective view showing a cut-away of a heart with a cardiac pump implanted into the left ventricle;

FIG. 2 is a perspective view of a cardiac pump with a section of the cardiac pump housing omitted for clarity;

FIG. 3 is a cross-section view of a cardiac pump;

FIG. 4 is a partial cross-sectional view of a magnetic bearing assembly for the cardiac pump shown in FIGS. 2 and 3;

FIG. 5 is a partial cross-sectional view of another magnetic bearing assembly for the cardiac pump;

FIG. 6 is a cross-section view of another cardiac pump;

FIG. 7 is a partial cross-sectional view of another magnetic bearing assembly for a cardiac pump; and

FIG. 8 is a cross-section view of another cardiac pump.

### DETAILED DESCRIPTION

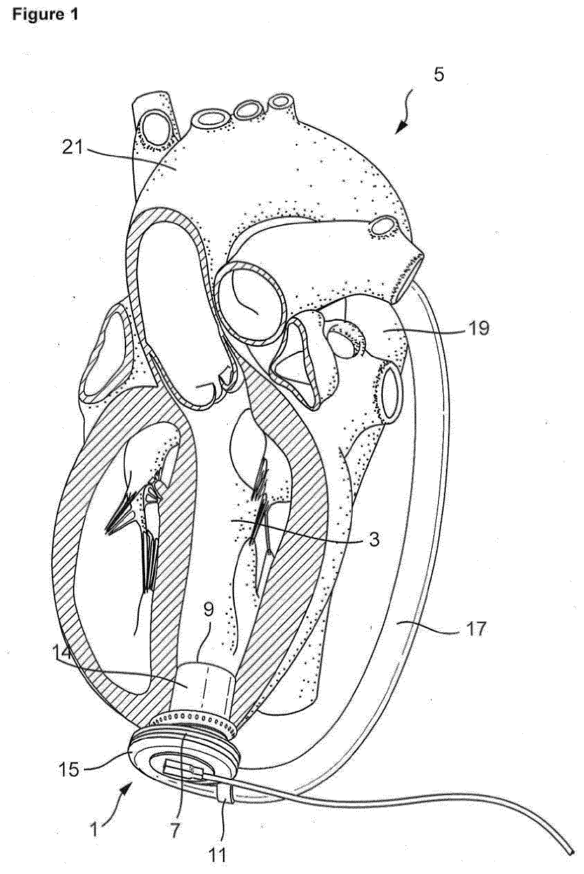

FIG. 1 depicts a blood pump, such as a cardiac pump 1, for the treatment of heart failure, for example a Ventricular Assist Device (VAD), in an implanted configuration in the left ventricle 3 of a heart 5. The cardiac pump 1 according to the present disclosure may be any appropriate type of cardiac pump. For example, the cardiac pump 1 may be an axial flow cardiac pump, a radial flow cardiac pump, or a mixed flow cardiac pump. It will be understood, therefore, that where technically possible, features described in relation to a radial flow cardiac pump may be employed in any type of cardiac pump, such as an axial flow cardiac pump, and vice versa. Further, whilst FIG. 1 depicts the cardiac pump in an implanted configuration in the left ventricle 3 of a heart 5, it is understood that the cardiac pump 1 may be implanted in any appropriate position, for example extracorporeally, completely outside of the heart 5, or completely inside of the heart 5.

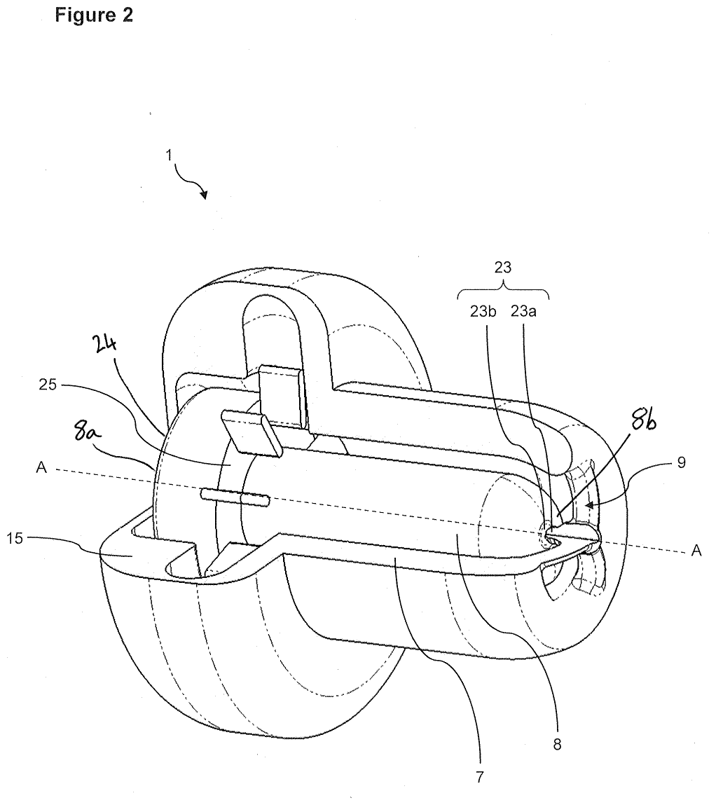

The cardiac pump 1 of FIG. 1 comprises a cardiac pump housing 7 comprising an inlet 9 for blood and an outlet 11 for blood. The cardiac pump 1 comprises a cardiac pump rotor 8 (depicted in FIG. 2) disposed at least partially within the cardiac pump housing 7. The cardiac pump rotor 8 is supported, for example rotatably supported, by way of one or more bearing assemblies 23, as described below.

The cardiac pump 1 may comprise an inflow tube, for example an inflow cannula 14, which may be integral to the cardiac pump housing 7. When the cardiac pump is in an implanted state, the inflow cannula 14 may be situated at least partially inside the left ventricle 3. A pumping chamber 15 may be situated outside of the heart 5. The inflow cannula 14 may be arranged to extend from the pumping chamber 15, through the wall of the left ventricle 3 into the left ventricle 3. In this way, the inlet 9 may be situated completely within the left ventricle 3. The pumping chamber 15 may be situated on the apex of the left ventricle 3 with the outlet 11 connected to a separate outflow cannula 17. In the example shown in FIG. 1, the outflow cannula 17 is anastomosed to a descending aorta 19, although in an alternative example the outflow cannula 17 may be anastomosed to an ascending aorta 21.

With reference to FIG. 2, the rotor 8 of the cardiac pump is supported at least partially within the cardiac pump housing 7. The cardiac pump rotor 8 comprises an impeller portion 25 which is configured to pump the blood. As depicted, the impeller portion 25 may be provided at or towards a first end 8a of the cardiac pump rotor 8.

The cardiac pump rotor 8 may be supported by one or more bearing assemblies. The bearing assemblies may be configured such that the cardiac pump rotor 8 is substantially constrained, e.g. in five degrees-of-freedom, and the cardiac pump rotor 8 can rotate about a rotational axis A-A. In other words, the one or more bearing assemblies of the cardiac pump 1 may permit rotation of the cardiac pump rotor 8, whilst sufficiently constraining to the cardiac pump rotor 8 in all other degrees of freedom. In this manner, the one or more bearing assemblies may support the cardiac pump rotor 8 in the axial and radial directions (relative to the rotation axis A-A), as well as in pitch and yaw.

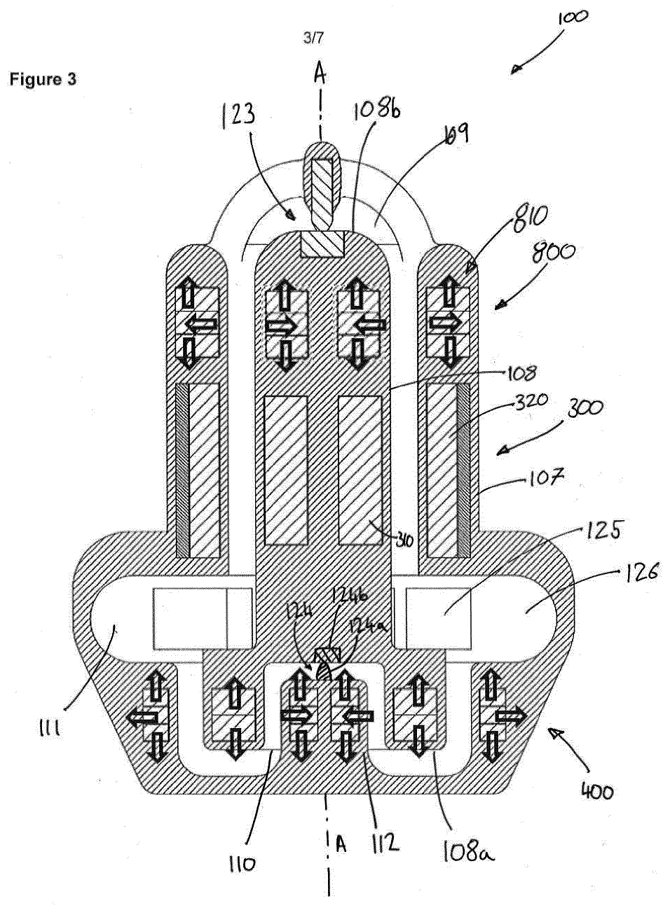

The cardiac pump 1 comprises one or more magnetic bearing assemblies 300, which are described in more detail below with reference to FIGS. 3 to 10. The cardiac pump rotor 8 may be further supported by a first plain bearing assembly 23, a second plain bearing assembly 24 and/or one or more electromagnetic bearing assemblies.

In the example shown in FIG. 2, the cardiac pump rotor 8 is partially supported by a first plain bearing assembly 23 located towards the inlet 9 of the cardiac pump, e.g. at or towards a second end 8b of the cardiac pump rotor. The plain bearing assembly 23 is a type of contact bearing assembly in which the bearing surfaces of the plain bearing assembly 23 are configured to be in contact during operation of the cardiac pump 1. For example, the plain bearing assembly 23 may comprise no intermediate rolling elements, i.e. motion is transmitted directly between two or more contacted surfaces of respective portions of the plain bearing assembly 23.

The first plain bearing assembly 23 comprises a first plain bearing portion 23a. The first plain bearing portion 23a is coupled to the cardiac pump housing 7 such that, during operation of the cardiac pump 1, the first plain bearing portion 23a does not rotate with the cardiac pump rotor 8. In FIGS. 2 and 3, the first plain bearing portion 23a is integral to the cardiac pump housing 7, although in an alternative example (not shown) the first plain bearing portion 23a may be a separate component rigidly fixed to the cardiac pump housing 7. In another example, the first plain bearing portion 23a may be movably coupled, for example threadably coupled, to the cardiac pump housing 7 such that the position of the first plain bearing portion 23a may be adjusted relative to the cardiac pump housing 7. The first plain bearing portion 23a may be constructed from a different material to the cardiac pump housing 7, e.g. a ceramic material. Alternatively, the first plain bearing portion 23a may be constructed from a similar material to the cardiac pump housing 7, e.g. a titanium alloy. The first plain bearing portion 23a may comprise a surface coating and/or may have had a surface treatment to improve the wear characteristics of the plain bearing assembly 23.

The plain bearing assembly 23 comprises a second plain bearing portion 23b. The second plain bearing portion 23b is coupled to the cardiac pump rotor 8 such that, during operation of the cardiac pump 1, the second plain bearing portion 23b rotates with the cardiac pump rotor 8. In the example shown in FIGS. 2 and 3, the second plain bearing portion 23b is integral to the cardiac pump rotor 8, although in an alternative example the second plain bearing portion 23b may be a separate component rigidly fixed to the cardiac pump rotor 8. In another example, the second plain bearing portion 23b may be movably coupled, for example threadably coupled, to the cardiac pump rotor 8 such that the position of the second plain bearing portion 23b may be adjusted relative to the cardiac pump rotor 8. The second plain bearing portion 23b may be constructed from a different material to the cardiac pump rotor 8, e.g. a ceramic material. Alternatively, the second plain bearing portion 23b may be constructed from a similar material to the cardiac pump rotor 8, e.g. a titanium alloy. The second plain bearing portion 23b may comprise a surface coating and/or may have had a surface treatment to improve the wear characteristics of the plain bearing assembly 23. The first and second plain bearing portions 23a, 23b may be constructed from different materials to each other, for example the first and second plain bearing portions 23a, 23b may each be constructed from a different ceramic material.

The first and second plain bearing portions 23a, 23b are configured to engage each other so as to be in contact when the cardiac pump rotor 8 and the cardiac pump housing 7 are in an assembled configuration, such that the plain bearing assembly 23 is configured to rotatably support the cardiac pump rotor 8 within the cardiac pump housing 7. In the example shown in FIGS. 2 to 4, the first and second bearing portions 23a, 23b each comprise a substantially planar articular bearing surface arranged perpendicularly to the rotational axis A-A. In this manner, the first and second bearing portions 23a, 23b are configured to at least partially support the cardiac pump rotor 8 within the cardiac pump housing 7 in an axial direction of the cardiac pump rotor 8.

The area of contact between the first and second bearing portions 23a, 23b may be optimised with regard to heat generation and wear characteristics of the plain bearing assembly 23. For example, the area of contact may be a substantially circular contact area having an appropriate diameter that may be selected dependent upon operational characteristics of the cardiac pump 1 and the material from which the first and/or second bearing portions 23a, 23b are fabricated. In one example, the substantially circular contact area may have a diameter within a range of approximately 10 μm to 3 mm, or, in particular, within a range of approximately 300 μm to 1 mm. It is appreciated, however, that the shape of the contact area may be of any appropriate form and/or size. In another example, the plain bearing assembly 23 may comprise a plurality of contact areas, which may each be optimised to provide the desired levels of heat generation and wear characteristics.

As described below, when a stiffness of a magnetic bearing assembly provided on the cardiac pump is relatively high, an axial preload on the plain bearing assembly 23 may be relatively high, and hence, the size and shape of the first and second bearing portions 23a, 23b may be sized and/or shaped in consideration of the high axial preload.

The cardiac pump rotor 8 depicted in FIGS. 1 and 2 further comprises a second plain bearing assembly 24 located towards the outlet 11 of the cardiac pump, e.g. at or towards a first end 8a of the cardiac pump rotor. The second plain bearing assembly 24 may be configured to act as an end stop for the cardiac pump rotor, e.g. which acts against the cardiac pump rotor in the event of the cardiac pump rotor become displaced towards the outlet end. The second plain bearing assembly 24 comprises a first and second plain bearing portions which may be configured in a similar way to the first and second plain bearing portions of the first plain bearing assembly described above.

With reference to FIG. 3, a cardiac pump 100, according to arrangements of the present disclosure will now be described. The cardiac pump 100 may be similar to the cardiac pump 1 described above with reference to FIGS. 1 and 2 and the features described in respect of the cardiac pump 1 may apply equally to the cardiac pump 100. In particular, the cardiac pump 100 comprises a rotor 108 and a housing 107, which are similar to the rotor and housing 8, 7 respectively. The cardiac pump 100 further comprises plain bearing assembly 123, which is similar to the plain bearing assembly 23. FIG. 3 is a cross-sectional view of a cardiac pump 100 according to arrangements of the present disclosure. Cross-sectional profiles of the components of the cardiac pump 100 may be constant at least partially around the rotational axis A-A of the cardiac pump. However, it will be appreciated that the shape of the components, such as the impeller portion 125 of the cardiac pump rotor 108 and an outlet volume 126 of the cardiac housing 107 may vary around the axis of rotation A-A

The cardiac pump rotor 108 extends along the rotational axis A-A of the cardiac pump 100 between first and second ends 108a, 108b of the rotor. As depicted, the first end 108a may be an outlet end of the rotor, e.g. at or closer to an outlet 111 of the cardiac pump 100 than the second end 108b. The second end 108b may be an inlet end of the rotor, e.g. at or closer to an inlet 109 of the cardiac pump 100 than the first end 108a. In the arrangement shown in FIG. 3, the housing 107, e.g. an external profile of the housing, and the inlet 109 are substantially symmetrically shaped about the rotational axis A-A of the cardiac pump 100. However, in other arrangements, the housing 107 may be any other shape. In particular, the housing 107 and/or the inlet 109 may not be symmetrical about the rotational axis A-A.

As depicted, the cardiac pump rotor 108 comprises a recess 110 formed in an axial end face of the cardiac pump rotor 108. In the arrangement shown, the recess 110 is formed in the axial end face at the outlet end 108a of the cardiac pump rotor. The recess 110 may be positioned centrally on the axial end face relative to the axis of rotation A-A. In other words, the recess 110 may extend radially outward from the axis of rotation A-A. In the arrangement depicted, the recess 110 is symmetrical about the axis of rotation A-A. For example, the recess 110 may be a cylindrically shaped recess having a central axis aligned with the axis of rotation A-A. In other arrangements, the recess may be any other shape and/or may be shaped non-symmetrically relative to, e.g. about, the axis of rotation A-A.

As illustrated the cardiac pump rotor may 108 extend from the inlet end 108b past the outlet 111, or at least part of the outlet volume 126, to the outlet end 108b of the cardiac pump rotor. In other words, the first end 108a of the cardiac pump rotor may be disposed on an opposite side of the outlet 111, or at least part of an outlet volume 126, of the cardiac pump rotor from the inlet end 108b of the cardiac pump rotor along the rotational axis A-A. The recess 110 may be formed, e.g. partially or substantially completely formed, in a portion of the cardiac pump rotor on an opposite side of the outlet, or at least a part of the outlet volume 126, from the inlet end 108b of the cardiac pump rotor.

The cardiac pump housing 107 further comprises a protruding portion 112, which extends axially, e.g. along the axis of rotation A-A, into the recess 110. The protruding portion 112 may be at least partially received within the recess 110.

As depicted in FIG. 3, the second plain bearing assembly 124 of the cardiac pump 100 may be arranged inside the recess 110. For example, a first plain bearing portion 124a of the second plain bearing assembly may be formed by the protruding portion 112 of the cardiac pump housing. The second plain bearing portion 124b of the second plain bearing assembly 124 may be formed inside the recess 110. The first and second plain bearing portions for the second plain bearing assembly may engage one another inside the recess 110.

As depicted in FIG. 3, the cardiac pump 100 comprises a first magnetic bearing assembly 400. Additionally or alternatively, the cardiac pump 100 may further comprise a second magnetic bearing assembly 800. The first and/or second magnetic bearing assemblies 400, 800 may be configured to support, e.g. radially support, the cardiac pump rotor 108 for rotation about the rotational axis A-A. The first and/or second magnetic bearing assemblies 400, 800 may be configured to operate in conjunction with the other bearing assemblies of the cardiac pump, such as the first and/or second plain bearing assemblies (if present) to support the cardiac pump rotor 108.

As depicted, the first magnetic bearing assembly 400 may be at least partially, or substantially completely, aligned with the recess 110 along the axis of rotation A-A of the cardiac pump rotor.

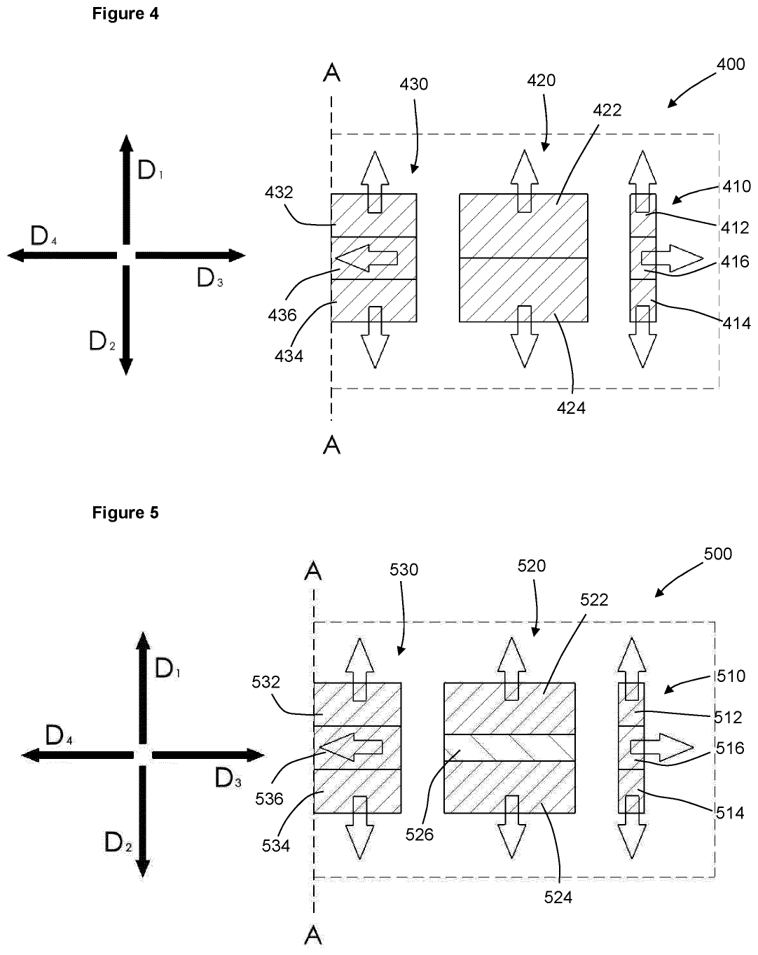

FIG. 4 is a partial sectional view of first magnetic bearing assembly 400 accordingly to an arrangement of the present disclosure. FIG. 4 illustrates the cross-sectional arrangement of the components of the first magnetic bearing assembly 400 to one side of the rotational axis A-A. The cross-sectional profiles of the components may be constant around the rotational axis A-A of the cardiac pump 100. Alternatively, the cross-sectional profiles of the components of the first magnetic bearing assembly 400 may vary, e.g. with angle, around the rotational axis A-A.

The first magnetic bearing assembly 400 comprises a first magnet assembly 410, a second magnet assembly 420 and a third magnet assembly 430. As depicted, the first magnet assembly 410 is provided on a portion of the cardiac pump housing 107 radially outward of the recess. The second magnet assembly 420 is provided on the rotor at a position radially outward of the protruding portion 112 of the cardiac pump housing. The third magnet assembly 430 is provided on the protruding portion 112 of the cardiac pump housing. The first, second and third magnet assemblies at least partially overlap one another in a direction parallel with the rotational axis A-A. In this way, the first, second and third magnet assemblies 410, 420, 430 may be configured to support, e.g. radially support, the rotor 108 for rotation about the rotational axis A-A.

As depicted in FIG. 4, the first magnet assembly 410 may comprise a plurality of magnets 412, 414, 416 arranged to produce a stronger magnetic field adjacent the first magnet assembly 410 in a radially inward direction and a weaker magnetic field adjacent to the first magnet assembly in a radially outward direction. For example the first magnet assembly 410 may comprise a half or full Halbach array of magnets, e.g. comprising, 3, more than 3 (such as 5) or more magnets extending along the rotational axis A-A. The half or full Halbach array of magnets may be configured to produce a stronger magnetic field to one side of the array of magnets in a radial direction relative to the other side. In particular, the half or full Halbach array of magnets may be configured to produce a stronger magnetic field to a side of the array of magnets adjacent the first magnet assembly 410 in a radially inward direction. The half or full Halbach array of magnets may be configured to produce a weaker magnetic field on the side of the Halbach array adjacent to the first magnet assembly 410 in a radially outward direction.

As shown in FIG. 4, the first magnet assembly 410 may comprise a first housing magnet 412, arranged such that a magnetic dipole moment of the first housing magnet is at least partially, e.g. substantially, aligned with a first direction D₁ parallel with the rotational axis. The first magnet assembly 410 may further comprise a second housing magnet 414. The second housing magnet may be adjacent to, or spaced apart from, the first housing magnet 412 along in the axial direction of the pump 100. The second housing magnet 414 may be arranged such that a magnetic dipole moment of the second housing magnet 414 is aligned with a second direction D₂ opposite the first direction. The first magnet assembly may further comprise a third housing magnet 416, arranged between the first and second magnets in the first and/or second direction, e.g. in an axial direction of the pump. The third housing magnet 416 may be arranged such that a magnetic dipole moment of the third housing magnet 416 is aligned with a third direction D₃ direction perpendicular to the first and second directions D₁, D₂.

The second magnet assembly 420 may comprise a first rotor magnet 422 and a second rotor magnet 424. The first and second rotor magnets 422, 424 may be arranged adjacent to one another, or spaced apart, in an axial direction of the pump 100. The first rotor magnet 422 may be arranged such that a magnetic dipole moment of the first rotor magnet is aligned with the first direction D₁. The second rotor magnet 424 may be arranged such that a magnetic dipole moment of the second rotor magnet is aligned with the second direction D₂ opposite the first direction. The second rotor magnet may be arranged immediately adjacent, e.g. in contact with, the first rotor magnet, e.g. without a pole piece or further magnet being provided between the first and second rotor magnets.

The third magnet assembly 430 may be similar to the first magnet assembly 410 and may comprise a plurality of magnets arranged to produce a stronger magnetic field adjacent the third magnet assembly to one side of the first magnet assembly, in a radial direction, than to the other side. The third magnet assembly 430 may differ from the first magnet assembly 410 in that the third magnet assembly may be configured to produce a stronger magnetic field adjacent the third magnet assembly in radially outward direction and a weaker magnetic field adjacent to the third magnet assembly in a radially inward direction.

The third magnet assembly 430 may comprises a half or full Halbach array of magnets extending along the rotational axis A-A. For example, the third magnet assembly 430 may comprise a fourth housing magnet 432, arranged such that a magnetic dipole moment of the fourth housing magnet is aligned with the first direction D₁ parallel with the rotational axis. The third magnet assembly 430 may further comprise a fifth housing magnet 434, arranged such that a magnetic dipole moment of the fifth housing magnet 434 is aligned with the second direction D₂ opposite the first direction. The third magnet assembly 430 may further comprise a sixth housing magnet 436, arranged between the fourth and fifth housing magnets in the first and/or second direction. The sixth housing magnet may be arranged such that a magnetic dipole moment of the sixth housing magnet 436 is aligned with a fourth direction D₄ direction perpendicular to the first and second directions. The fourth direction D₄ may be opposite the third direction D₃.

Due to the configurations of the first, second and third magnet assemblies 410, 420, 430, described above, there may be a large magnetic field strength in gaps between the first magnet assembly 410 and the second magnet assembly 420, and between the third magnet assembly 430 and the second magnet assembly 420. Due to the large field strength in these areas, a stiffness of the first magnetic bearing assembly 400 may be greater than the stiffness of magnetic bearing assemblies in prior art arrangements. A stiffness of the first magnetic bearing assembly may be greater than 6 N/mm. For example, a stiffness of the first magnetic bearing assembly may be approximately 13 N/mm. Due to the high stiffness of the first magnetic baring assembly, the cardiac pump 100 may be configured to operate effectively at sub-critical rotor speeds, as opposed to super critical speeds for pumps or miniaturised pumps without such bearing assemblies.

With reference to FIG. 5, a first magnetic bearing assembly 500 according to another arrangement will now be described, the first magnetic bearing assembly 500 depicted in FIG. 5 may be provided as part of the cardiac pump 100 depicted in FIG. 3, e.g. in place of the first magnetic bearing assembly 400. FIG. 5 illustrates the cross-sectional arrangement of the components of the first magnetic bearing assembly 500 to one side of the rotational axis A-A. The cross-sectional profiles of the components may be constant around the rotational axis A-A of the cardiac pump 100. Alternatively, the cross-sectional profiles of the components of the first magnetic bearing assembly 500 may vary, e.g. with angle, around the rotational axis A-A.

The first magnetic bearing assembly 500 comprises a first magnet assembly 510, a second magnet assembly 520 and a third magnet assembly 530.

The first magnet assembly 510 may be similar to the first magnet assembly 410 described above, and the features described in relation to the first magnet assembly 410 may apply equally to the first magnet assembly 510. In particular, the first magnet assembly 510 may comprise first, second and third housing magnets 512, 514, 516, which may be arranged in a similar way to the first, second and third housing magnets 412, 414, 416 of the first magnet assembly 410.

The third magnet assembly 530 may also be similar to the third magnet assembly 430 described above, and the features described above in relation to the third magnet assembly 430 may apply equally to the third magnet assembly 530. In particular, the third magnet assembly 530 may comprise fourth, fifth and sixth housing magnets 532, 534, 536, which may be arranged in a similar way to the first, second and third housing magnets 432, 434, 436 of the third magnet assembly 430.

The first magnetic bearing assembly 500 differs from the first magnetic bearing assembly 400 in that the second magnet assembly 520 comprises a first rotor magnet 522, a second rotor magnet 524 and a pole piece 526 arranged between the first and second rotor magnets, e.g. along the axis of rotation A-A. The pole piece 526 may be made from a material with a high magnetic permeability. The first rotor magnet 522 may be arranged such that a magnetic dipole moment of the first rotor magnet is aligned with the first direction D₁. The second rotor magnet 524 may be arranged such that a magnetic dipole moment of the second rotor magnet is aligned with the second direction D₂ opposite the first direction. The first and second rotor magnets 522, 524 may be arranged in contact with the pole piece 526 on opposite sides of the pole piece in the first and/or second direction D₁, D₂.

Due to the configurations of the first, second and third magnet assemblies 510, 520, 530, described above, there may be a large magnetic field strength in gaps between the first magnet assembly 510 and the second magnet assembly 520, and between the third magnet assembly 530 and the second magnet assembly 520. Due to the large field strength in these areas, a stiffness of the first magnetic bearing assembly 500 may be greater than in prior art arrangements. Further, the magnetic field strength in the gaps between the first magnet assembly and the second magnet assembly, and between the third magnet assembly and the second magnet assembly may be greater than the magnetic field strength in corresponding locations of the arrangement depicted in FIG. 4. The stiffness of the first magnetic bearing assembly 500 may therefore be greater than the stiffness of the first magnetic bearing assembly 400. A stiffness of the first magnetic bearing assembly 500 may be greater than 13 N/mm. For example, a stiffness of the first magnetic bearing assembly 500 may be approximately 15 N/mm. Accordingly, due to the high stiffness of the first magnetic baring assembly 500, the cardiac pump 100 may be configured to operate effectively at sub-critical rotor speeds.

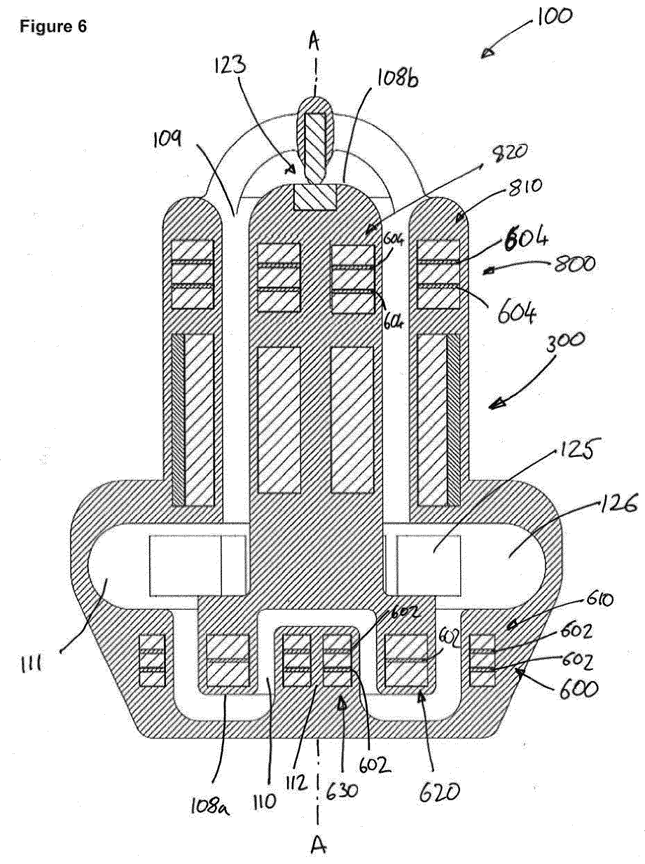

With reference to FIG. 6, a first magnetic bearing assembly 600 according to another arrangement of the present disclosure will now be described. As illustrated, the first magnetic bearing assembly 600 may be provided as part of the cardiac pump 100, e.g. in place of the first magnetic bearing assembly 400. FIG. 6 is a cross-sectional view of a cardiac pump 100 according to arrangements of the present disclosure. It will be appreciated that cross-sectional profiles of respective ones of the components of the cardiac pump 100 may be constant at least partially around the rotational axis A-A of the cardiac pump and/or may vary around the rotational axis.

The first magnetic bearing assembly 600 may be similar to the first magnetic bearing assembly 400 and the features described in relation to first magnetic bearing assembly 400 may apply equally to the first magnetic bearing assembly 600. The first magnetic bearing assembly 600 may differ from the first magnetic bearing assembly 300, in that one or more respective pole pieces 602 may be provided between one more adjacent pairs of magnets within the first, second and/or third magnet assemblies 610, 620, 630.

In the arrangement shown in FIG. 6, pole pieces 602 are provided between each of the adjacent magnets within the first, second and third magnet assemblies 610, 620, 630, e.g. in a direction parallel with the rotational axis A-A. However, in other arrangements one or more of the pole pieces 602 illustrated in FIG. 6 may be omitted.

A stiffness of the first magnetic bearing assembly 600 may be greater than 13 N/mm and may be greater than 15 N/mm. Accordingly, due to the high stiffness of the first magnetic baring assembly 600, the cardiac pump 100 may be configured to operate effectively at sub critical rotor speeds.

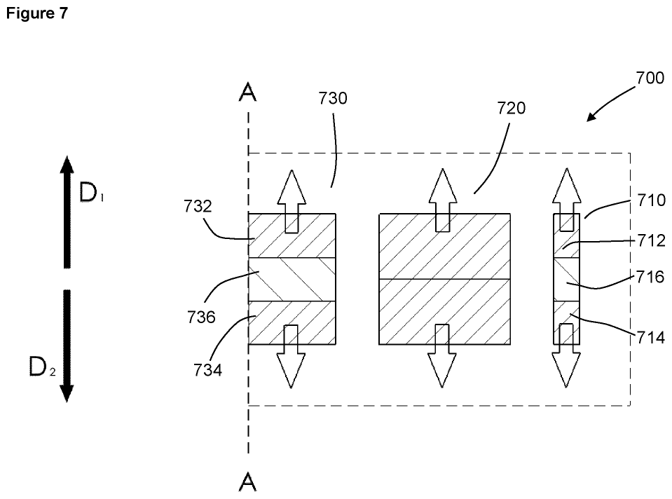

With reference to FIG. 7, a first magnetic bearing assembly 700 according to another arrangement of the present disclosure will now be described, the first magnetic bearing assembly 700 depicted in FIG. 7 may be provided as part of the cardiac pump 100 depicted in FIG. 3 or 6, e.g. in place of the first magnetic bearing assembly 400, 600. FIG. 7 illustrates the cross-sectional arrangement of the components of the first magnetic bearing assembly 700 to one side of the rotational axis A-A. The cross-sectional profiles of the components may be constant around the rotational axis A-A of the cardiac pump 100. Alternatively, the cross-sectional profiles of the components of the first magnetic bearing assembly 700 may vary, e.g. with angle, around the rotational axis A-A.

The first magnetic bearing assembly 700 comprises a first magnet assembly 710, a second magnet assembly 720 and a third magnet assembly 730. In the same way as the first, second and third magnet assemblies 410, 610, 420, 620, 430, 630 of the first magnetic bearing assemblies 400, 600 described above, the first magnet assembly 710 is provided on a portion of the cardiac pump housing 107 radially outward of the recess 110. The second magnet assembly 720 is provided on the rotor at a position radially outward of the protruding portion 112 of the cardiac pump housing. The third magnet assembly 730 is provided on the protruding portion 112 of the cardiac pump housing. The first, second and third magnet assemblies at least partially overlap one another in a direction parallel with the rotational axis A-A.

The second magnet assembly 720 of the first magnetic bearing assembly 700 may be similar to the second magnet assembly 520 of the first magnetic bearing assembly 500 described above with reference to FIG. 5. The features described in relation to the second magnet assembly 520 may apply equally to the second magnet assembly 720.

The first magnet assembly 710 comprises a first housing magnet 712, a second housing magnet 714 and a first housing pole piece 716 arranged between the first and second housing magnets 714, 716, e.g. along the rotational axis A-A. The pole piece 716 may made from a material with a high magnetic permeability, such as iron, e.g. soft or annealed iron. As depicted, the first housing magnet 712 may be arranged such that a magnetic dipole moment of the first housing magnet is aligned with the first direction D₁. The second housing magnet 714 may be arranged such that a magnetic dipole moment of the second rotor magnet is aligned with the second direction D₂ opposite the first direction. The first and second housing magnets 712, 714 may be arranged in contact with the pole piece 716 on opposite sides of the pole piece in the first and/or second direct D₁, D₂.

The third magnet assembly 730 comprises a third housing magnet 732, a fourth housing magnet 734 and a second housing pole piece 736 arranged between the third and fourth housing magnets 732, 734, e.g. along the rotational axis A-A. The pole piece 736 may made from a material with a high magnetic permeability, such as iron, e.g. soft or annealed iron. As depicted, the third housing magnet 732 may be arranged such that a magnetic dipole moment of the first housing magnet is aligned with the first direction D₁. The fourth housing magnet 734 may be arranged such that a magnetic dipole moment of the second rotor magnet is aligned with the second direction D₂ opposite the first direction. The third and fourth housing magnets 732, 734 may be arranged in contact with the pole piece 736 on opposite sides of the pole piece in the first and/or second direct D₁, D₂.

Due to the configurations of the first, second and third magnet assemblies 710, 720, 730, described above, there may be a large magnetic field strength in gaps between the first magnet assembly 710 and the second magnet assembly 720, and between the third magnet assembly 730 and the second magnet assembly 720. Due to the large field strength in these areas, a stiffness of the first magnetic bearing assembly 700 may be greater than in prior art arrangements. The magnetic field strength in the gaps between the first magnet assembly 710 and the second magnet assembly 720, and between the third magnet assembly 730 and the second magnet assembly 720 may be less than the magnetic field strength in corresponding locations of the arrangement depicted in FIGS. 4, 5 and 6. The stiffness of the first magnetic bearing assembly 700 may therefore be less than the stiffness of the first magnetic bearing assembly 400, 600. For example, a stiffness of the first magnetic bearing assembly 600 may be less than 15 N/mm, such as between 12 N/mm and 15 N/mm. Nevertheless, due to the high stiffness of the first magnetic baring assembly, the cardiac pump 100 may be configured to operate effectively at sub critical rotor speeds.

Returning briefly to FIG. 3, the second magnetic bearing assembly 800 may be provided at or towards the second end 108b of the rotor. In particular, the second magnetic bearing assembly 800 may be provided closer to the second end 108b of the rotor than the first magnetic bearing assembly 400.

The second magnetic bearing assembly 800 may comprise a fourth magnet assembly 810 and a fifth magnet assembly 820. The fourth magnet assembly may be provided on the housing 107 and the fifth magnet assembly 820 may be provided on the rotor. The fourth and fifth magnet assemblies 810, 820 may at least partially overlap one another in a direction parallel with the rotational axis A-A.

The fourth magnet assembly 810 may comprise a plurality of magnets 812, 814, 816 arranged to produce a stronger magnetic field adjacent the fourth magnet assembly 810 in a radially inward direction and a weaker magnetic field adjacent to the fourth magnet assembly in a radially outward direction. For example the fourth magnet assembly 810 may comprise a half or full Halbach array of magnets, e.g. comprising, 3, more than 3 (such as 5) or more magnets extending along the rotational axis A-A. The half or full Halbach array of magnets may be configured to produce a stronger magnetic field to one side of the array of magnets in a radial direction relative to the other side. In particular, the half or full Halbach array of magnets may be configured to produce a stronger magnetic field to a side of the array of magnets adjacent the fourth magnet assembly 810 in a radially inward direction. The half or full Halbach array of magnets may be configured to produce a weaker magnetic field on the side of the Halbach array adjacent to the fourth magnet assembly 810 in a radially outward direction.

As depicted in FIG. 3, the fifth magnet assembly 820 may comprise a plurality magnets arranged to produce a stronger magnetic field adjacent the fifth magnet assembly 820 in a radially outward direction and a weaker magnetic field adjacent to the fourth magnet assembly in a radially inward direction. For example the fifth magnet assembly 820 may comprise a half or full Halbach array of magnets, e.g. comprising, 3, more than 3 (such as 5) or more magnets extending along the axis of rotation A-A. The half or full Halbach array of magnets may be configured to produce a stronger magnetic field to one side of the array of magnets in a radial direction relative to the other side. In particular, the half or full Halbach array of magnets may be configured to produce a stronger magnetic field to a side of the array of magnets adjacent the fifth magnet assembly 820 in a radially outward direction. The half or full Halbach array of magnets may be configured to produce a weaker magnetic field on the side of the Halbach array adjacent to the fifth magnet assembly 820 in a radially inward direction.

Referring now to FIG. 6, in some arrangements, one or more pole respective pole pieces 604 may be provided between adjacent ones of the magnets provided within the fourth and/or fifth magnet assemblies 810, 820, e.g. in a direction parallel with the axis of rotation A-A. In the arrangement depicted in FIG. 6, pole pieces are provided between each pair of adjacent magnets within the fourth and fifth magnet assemblies 810, 820. However in other arrangements, one or more of the pole pieces may be omitted and the adjacent magnets may be arranged adjacent, e.g. immediately adjacent or in contact with one another, with no pole piece in between,

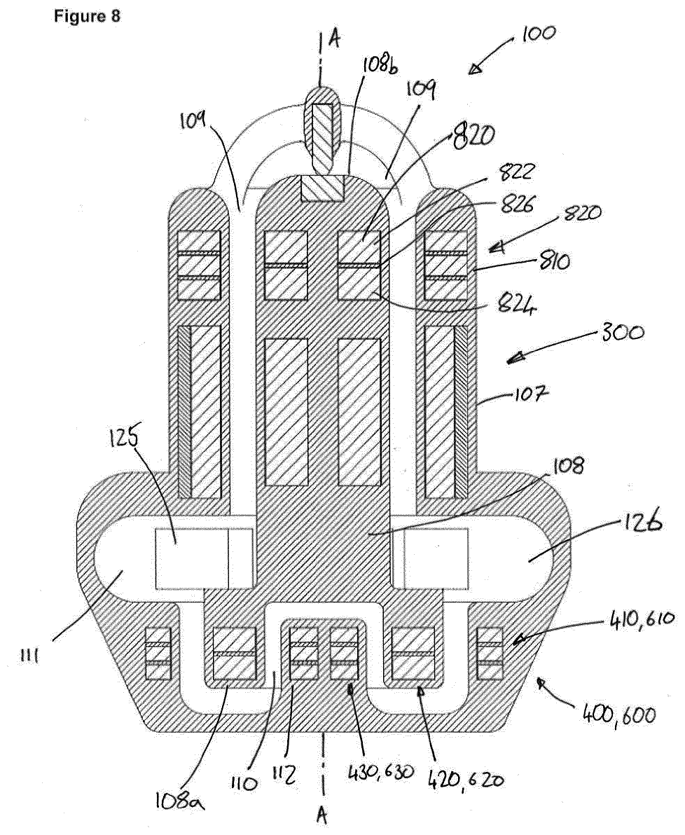

FIG. 8 is a cross-sectional view of the cardiac pump 100 according to arrangements of the present disclosure. It will be appreciated that cross-sectional profiles of respective ones of the components of the cardiac pump 100 may be constant at least partially around the rotational axis A-A of the cardiac pump and/or may vary around the rotational axis. As depicted in FIG. 8, in some arrangements, the fifth magnet assembly 820 may comprise a third rotor magnet 822, a fourth rotor magnet 824 and a pole piece 826 provided between the fourth and fifth rotor magnets 822, 824. As depicted, the third and fourth rotor magnet may stacked, e.g. arranged adjacent to one another or spaced apart, in a direction parallel with the rotational axis A-A of the rotor. The third rotor magnet 822 may be arranged such that a magnetic dipole moment of the third rotor magnet 822 is aligned with the first direction D₁. The fourth rotor magnet 824 may be arranged such that a magnetic dipole moment of the fourth rotor magnet is aligned with the second direction D₂. In some arrangements, the pole piece 826 may be omitted.

Referring to FIGS. 3, 6 and 8, the cardiac pump 100 may comprise a magnetic drive assembly 300, for example a brushless DC motor. As depicted, the cardiac pump rotor 108 may comprise a first portion 310 of the magnetic drive coupling, for example one or more permanent magnets. The cardiac pump housing 107 may comprise a second portion 320 of the magnetic drive coupling, for example one or more electrical windings. The magnetic drive coupling may be a radial magnetic drive coupling, e.g. a radial flux gap electric motor, although it is appreciated that the magnetic drive coupling may be of any appropriate configuration. As depicted, the magnetic drive assembly 300 may be arranged between the first and second magnetic bearing assemblies 400, 800 along the rotational axis A-A.

It will be appreciated by those skilled in the art that although the invention has been described by way of example, with reference to one or more exemplary examples, it is not limited to the disclosed examples and that alternative examples could be constructed without departing from the scope of the invention as defined by the appended claims.

1. A cardiac pump comprising:
a housing;
a rotor extending along a rotational axis of the cardiac pump between first and second axial ends of the rotor; and
a bearing assembly, wherein the bearing assembly is configured to support the rotor within the cardiac pump housing for rotation about the rotational axis, wherein the rotor comprises a recess formed in an axial end face of the rotor, wherein the housing comprises a protruding portion extending axially into the recess, and wherein the bearing assembly comprises:
a first magnet assembly provided on a portion of the housing radially outward of the recess formed in the rotor;
a second magnet assembly provided on a portion of the rotor radially outward of the protruding portion of the housing; and
a third magnet assembly provided on the protruding portion of the housing, wherein the first, second and third magnet assemblies at least partially overlap one another in a direction parallel with the rotational axis.

2. The cardiac pump of claim 1, wherein the first axial end of the rotor is an outlet end and the second axial end of the rotor is an inlet end, wherein the recess is formed in the axial end face at the outlet end of the rotor.

3. The cardiac pump of claim 1, wherein the first magnet assembly comprises a plurality of magnets arranged to produce a stronger magnetic field adjacent the first magnet assembly in a radially inward direction and a weaker magnetic field adjacent to the first magnet assembly in a radially outward direction.

4. The cardiac pump of claim 1, wherein the first magnet assembly comprises a half or full Halbach array of magnets arranged to produce a stronger magnetic field adjacent the first magnet assembly in a radially inward direction and a weaker magnetic field adjacent to the first magnet assembly in a radially outward direction.

5. The cardiac pump of claim 1, wherein the first magnet assembly comprises:
a first housing magnet, arranged such that a magnetic dipole moment of the first housing magnet is aligned with a first direction parallel with the rotational axis; and
a second housing magnet, arranged such that a magnetic dipole moment of the second magnet is aligned with a second direction opposite the first direction.

6. The cardiac pump of claim 5, wherein the first magnet assembly comprises a pole piece arranged between the first and second housing magnets.

7. (canceled)

8. The cardiac pump of claim 1, wherein the second magnet assembly comprises.
a first rotor magnet, arranged such that a magnetic dipole moment of the first magnet is aligned with a first direction parallel with the rotational axis; and
a second rotor magnet adjacent the first rotor magnet, arranged such that a magnetic dipole moment of the second magnet is aligned with a second direction opposite the first direction.

9. The cardiac pump of claim 1, wherein the second magnet assembly comprises:
a first rotor magnet, arranged such that a magnetic dipole moment of the first rotor magnet is aligned with a first direction parallel with the rotational axis;
a second rotor magnet, arranged such that a magnetic dipole moment of the second rotor magnet is aligned with a second direction opposite the first direction; and
a pole piece arranged between the first and second rotor magnets.

10. The cardiac pump of claim 1, wherein the third magnet assembly comprises a plurality of magnets arranged to produce a stronger magnetic field adjacent the third magnet assembly in a radially outward direction and a weaker magnetic field adjacent to the third magnet assembly in a radially inward direction.

11. The cardiac pump of claim 1, wherein the third magnet assembly comprises a half or full Halbach array of magnets arranged to produce a stronger magnetic field adjacent the third magnet assembly in a radially outward direction and a weaker magnetic field adjacent to the third magnet assembly in a radially inward direction.

12. The cardiac pump of claim 1, wherein the third magnet assembly comprises;
a fourth housing magnet, arranged such that a magnetic dipole moment of the fourth housing magnet is aligned with a first direction parallel with the rotational axis; and
a fifth housing magnet, arranged such that a magnetic dipole moment of the fifth housing magnet is aligned with a second direction opposite the first direction.

13. (canceled)

14. The cardiac pump of claim 1, wherein the bearing assembly further comprises a fourth magnet assembly provided on the housing and a fifth magnet assembly provided on the rotor, wherein the fourth and fifth magnet assemblies are spaced apart from the recess in the rotor in a direction parallel with the rotational axis, and wherein the fourth and fifth magnet assemblies at least partially overlap one another in a direction parallel with the rotational axis.

15. (canceled)

16. The cardiac pump of claims 14, wherein the cardiac pump further comprises a magnetic drive assembly comprising one or more drive magnets for driving rotation of the rotor relative the housing, wherein the one or more drive magnets are arranged between the first, second and third magnet assemblies, and the fourth and fifth magnet assemblies.

17. The cardiac pump of claim 14, wherein the fifth magnet assembly comprises:
a third rotor magnet, arranged such that a magnetic dipole moment of the third magnet is aligned with a first direction parallel with the rotational axis; and
a fourth rotor magnet adjacent the third rotor magnet, arranged such that a magnetic dipole moment of the fourth rotor magnet is aligned with a second direction opposite the first direction.

18. The cardiac pump of claim 14, wherein the fifth magnet assembly comprises:
a third rotor magnet, arranged such that a magnetic dipole moment of the third magnet is aligned with a first direction parallel with the rotational axis; and
a fourth rotor magnet, arranged such that a magnetic dipole moment of the second magnet is aligned with a second direction opposite the first direction; and a pole piece arranged between the third and fourth rotor magnets.

19. The cardiac pump of claim 14, wherein the fourth magnet assembly comprises a plurality of magnets arranged to produce a stronger magnetic field adjacent the fourth magnet assembly in a radially inward direction and a weaker magnetic field adjacent to the fourth magnet assembly in a radially outward direction.

20. The cardiac pump of claim 14, wherein the fourth magnet assembly comprises a half or full Halbach array of magnets arranged to produce a stronger magnetic field adjacent the fourth magnet assembly in a radially inward direction and a weaker magnetic field adjacent to the fourth magnet assembly in a radially outward direction.

21. (canceled)

22. The cardiac pump of claim 14, wherein the fifth magnet assembly comprises a plurality of magnets arranged to produce a stronger magnetic field adjacent the fifth magnet assembly in a radially outward direction and a weaker magnetic field adjacent to the fifth magnet assembly in a radially inward direction.

23. (canceled)

24. The cardiac pump of claim 1, wherein the protruding portion of the housing is configured to engage the rotor at the recess to form a plane bearing between the housing and the rotor at the first end of the rotor.

25. The cardiac pump of claim 1, wherein the housing is configured to engage the rotor at the second end of the rotor to form a further plane bearing between the housing and the rotor at the second end of the rotor.
